(19) United States
(12) Patent Application Publication
(10) Pub. No.: US 2013/0289396 A1
(43) Pub. Date: Oct. 31, 2013

(54) OCT PROBE

(71) Applicants: Masashi Kitatsuji, Tokyo (JP); Seiichi Yokoyama, Tokyo (JP); Yoshiyuki Tashiro, Tokyo (JP)

(72) Inventors: Masashi Kitatsuji, Tokyo (JP); Seiichi Yokoyama, Tokyo (JP); Yoshiyuki Tashiro, Tokyo (JP)

(73) Assignee: HOYA CORPORATION, Tokyo (JP)

(21) Appl. No.: 13/997,821

(22) PCT Filed: Jan. 13, 2012
(86) PCT No.: PCT/JP2012/050546

§ 371 Date: Jun. 25, 2013

(30) Foreign Application Priority Data
Jan. 19, 2011 (JP) 2011-008815
Jan. 19, 2011 (JP) 2011-008816

(51) Int. Cl. A61B 5/00

(52) U.S. Cl. CPC A61B 5/0066
USPC 600/425

(57) ABSTRACT

There is provided an OCT probe, comprising: a flexible tube; an optical fiber that transmits object light and is supported in the flexible tube to be able to freely rotate about an axis of the optical fiber; an objective optical system that is fixed to a tip of the optical fiber and includes a condensing optical system which condenses the object light emerging from the optical fiber, and a deflection optical element which irradiates a subject with the object light by deflecting the condensed object light; and a barycenter adjustment member that is fixed to the objective optical system and causes a combined barycenter of the objective optical system and the barycenter adjustment member to be situated on the axis of the optical fiber.

### TECHNICAL FIELD

The present invention relates an OCT (Optical Coherence Tomography) probe for shooting a tomographic image near a surface layer of a lumen.

### BACKGROUND ART

As an observation system for observing in detail a fine structure near a surface layer of a lumen, such as a digestive organ or a bronchial tube, an OCT system is being put to practical use. An example of a specific configuration of an OCT system of this type is described, for example, in Japanese Patent Publications Nos. JP3628026B (hereafter, referred to as patent document 1) and JP4021975B (hereafter, referred to as patent document 2).

The OCT system includes an OCT probe to be inserted into a lumen. The OCT probe described in each of the patent documents 1 and 2 irradiates a subject with low coherence light by transmitting the low coherence light emitted from a light source through an optical fiber. In accordance with rotation of the optical fiber about an axis thereof, the low coherence light scans on the subject in a circumferential direction. The OCT system measures how much and where scanning light is reflected and scattered on the subject based on the principle of low coherence interferometry, and calculates and generates image data near a surface layer of the subject using measurement results. The generated image near the surface layer has a higher magnification and a higher resolution than those of an observation image generated by a normal electronic scope or a normal fiber scope.

Since the optical fiber for transmitting the low coherence light is long and is able to bend along a shape of a lumen into which the optical fiber is inserted, the optical fiber is warped and twisted in a sheath. Therefore, a rotation torque produced by a rotation and drive mechanism coupled to a proximal side of the optical fiber is not smoothly transmitted to a tip portion of the optical fiber. When transmission of the rotation torque is not smooth, the rotation speed of a deflection prism attached to the tip portion of the optical fiber fluctuates and thereby the scanning speed becomes irregular. As a result, the precision of a generated tomographic image decreases. For this reason, the OCT probe described in each of the patent documents 1 and 2 is configured such that a torque wire (a torque cable and a flexible shaft) is arranged around the optical fiber so that the rotation torque on the proximal side is steadily transmitted to the tip portion.

### SUMMARY OF THE INVENTION

The optical fiber which transmits the low coherence light is arranged such that the proximal side thereof is coupled to the rotation and drive mechanism and thereby the proximal side is supported approximately along the axis. However, there is no component that supports the tip side of the optical fiber. The optical fiber is supported in the sheath in a state of a long cantilever beam. Therefore, when the optical fiber is rotated by driving the rotation and drive mechanism, the tip portion of the optical fiber produces a swinging motion in the sheath. At the tip of the optical fiber, an optical component, such as a deflection prism, is fixed. Such a configuration also raises a problem that the weight of the optical component amplifies the swinging motion of the tip portion. If the optical fiber produces the swinging motion, the position of the deflection prism changes. Therefore, a problem arises that a focal point becomes unstable and undulates, and thereby it becomes impossible to obtain a fine tomographic image.

The present invention is made in view of the above described circumstances. The object of the invention is to provide an OCT probe suitable for suppressing a swinging motion of a tip portion of an optical fiber.

To solve the above described problem, according to an embodiment of the invention, there is provided an OCT probe, comprising: a flexible tube; an optical fiber that transmits object light and is supported in the flexible tube to be able to freely rotate about an axis of the optical fiber; an objective optical system that is fixed to a tip of the optical fiber and includes a condensing optical system which condenses the object light emerging from the optical fiber, and a deflection optical element which irradiates a subject with the object light by deflecting the condensed object light; and a barycenter adjustment member that is fixed to the objective optical system. Since the barycenter adjustment member causes a combined barycenter of the objective optical system and the barycenter adjustment member to be situated on the axis of the optical fiber so that a rotation center axis of a tip portion of the optical fiber becomes stable.

By causing the tip portion of the optical fiber to rotate stably about the axis thereof, the position of the objective optical system is also made stable on the same axis. As a result, the focal point becomes stable, and such a configuration is advantageous in obtaining a fine tomographic image.

The condensing optical system, the deflection optical element and the barycenter adjustment member are made of, for example, a same material or of materials having a same specific gravity.

The deflection optical element may be a deflection prism which is formed such that at least an end of a column is cut by a plane forming a certain angle with respect to an axis direction and a cut surface of the column is processed to be a reflection surface. The barycenter adjustment member may be formed such that: the barycenter adjustment member is based on a cylindrical shape having substantially a same diameter as that of the deflection prism; a tip of the barycenter adjustment member has a semispheric shape; and the barycenter adjustment member has a proximal end surface formed to be cut by a plane forming the certain angle with respect to an axis direction of the barycenter adjustment member. For example, the proximal end surface is adhered and fixed to a back side of the reflection surface so that the deflection prism and the barycenter adjustment member become coaxial with each other. With this configuration, no edge appears on an outer shape contour. Therefore, there is no part having a large fluid resistance during rotational motion, and thereby occurrence of cavitation can be effectively suppressed.

Preferably, at least a part of an outer circumferential surface of the optical fiber is covered with a fluorocarbon resin coat. In this case, it is preferable that the fluorocarbon resin coat is a PTF (Polytetrafluoroethylene) coat or a multilayer coat in which a PI (Polyimide) coat and a PFA (Polyfluoroalkoxy) coat overlap with each other. With this configuration, the frictional resistance between the optical fiber and the flexible tube decreases. Therefore, even if the optical fiber contacts an inner circumferential surface of the flexible tube during the rotational motion, loss of torque is small and the optical fiber is able to smoothly rotate.

According to the invention, an OCT probe suitable for suppressing a swinging motion of a tip portion of an optical fiber is provided.

### BRIEF DESCRIPTION OF THE DRAWINGS

FIG. 1 is a block diagram illustrating a configuration of an OCT system according to an embodiment of the invention.

FIG. 2 illustrates an internal configuration of an OCT probe according to example 1 of the invention.

FIG. 3 illustrates an internal configuration of an OCT probe according to example 2 of the invention.

FIG. 4 illustrates an internal configuration of an OCT probe according to example 3 of the invention.

### EMBODIMENTS FOR CARRYING OUT THE INVENTION

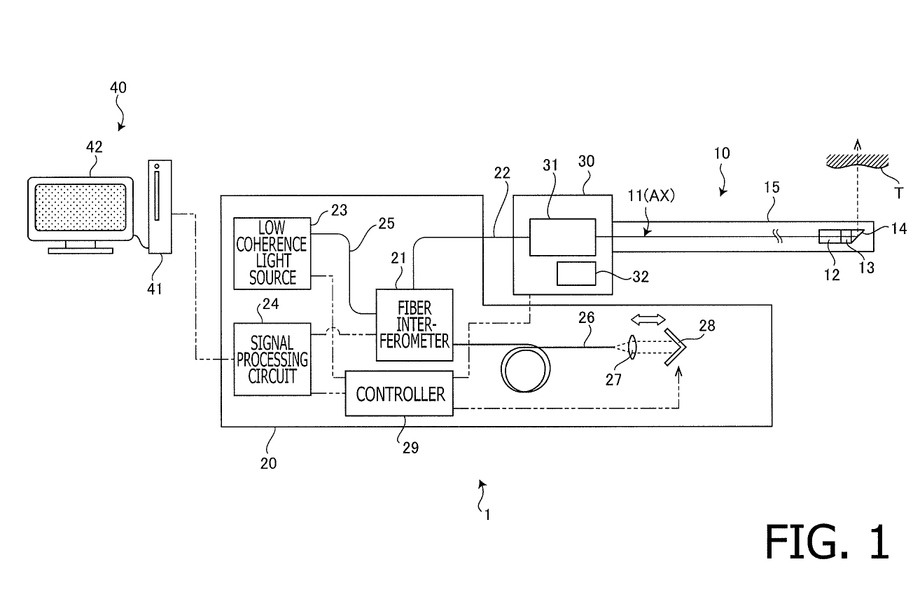

In the following, an OCT system according to an embodiment of the invention is explained with reference to the accompanying drawings. FIG. 1 is a block diagram generally illustrating a configuration of an OCT system 1 according to the embodiment. In FIG. 1, a path of an electric signal is represented by a double chain line, an optical path of an optical fiber is represented by a solid line and an optical path of light proceeding through air or a living tissue is represented by a dashed line. In the following explanation, in regard to an optical path in the OCT system 1, a side closer to a light source is defined as a proximal side, and a side farther from the light source is defined as a tip side.

As shown in FIG. 1, the OCT system 1 has an OCT probe 10 for obtaining an image near a surface layer of a lumen T, such as a digestive organ or a bronchial tube. The OCT probe 10 is connected to a system main unit 20 via a probe scanning device 30. Specifically, the probe scanning device 30 optically connects a proximal end of an optical fiber 11 of the OCT probe 10 with a tip of a probe optical fiber 22 extending to the outside of the system main unit 20 from a fiber interferometer 21 of the system main unit 20. In FIG. 1, for convenience of explanation, a configuration of the OCT probe 10 is represented by minimum elements required for explaining the principle of OCT observation. Furthermore, for convenience of explanation, the center axis (which coincides with the rotation center axis of the optical fiber 11 in design) of the OCT probe 10 is referred to as a “reference axis AX”.

In addition to the fiber interferometer 21 and the probe optical fiber 22, the system main unit 20 has a low coherence light source 23, a signal processing circuit 24, a supply optical fiber 25, a reference optical fiber 26, a lens 27, a dach mirror 28 and a controller 29. The controller 29 totally executes various types of control of the OCT system 1, such as light emission control of the low coherence light source 23, control of the signal processing circuit 24 and driving of motors for the dach mirror 28 and the probe scanning device 30.

The low coherence light source 23 is a light source being able to emit low coherence light, and specifically the low coherence light source 23 is a SLD (Super Luminescent Diode). The low coherence light emitted from the low coherence light source 23 is incident on the proximal end of the supply optical fiber 25. The supply optical fiber 25 transmits the low coherence light being incident thereon to the fiber interferometer 21. The fiber interferometer 21 divides the low coherence light from the supply optical fiber 25 into two optical paths with an optical coupler. One of the divided optical paths propagates through the probe optical fiber 22 as object light. The other of the divided optical paths propagates through the reference optical fiber 26 as reference light.

The probe scanning device 30 has a rotary joint 31 which couples the tip of the probe optical fiber 22 with the proximal end of the optical fiber 11. To the rotary joint 31, a radial scan motor 32 is connected via a transmission mechanism not shown. In accordance with driving of the radial scan motor 32, the rotary joint 31 rotates the optical fiber 11 about the reference axis AX, with respect to the probe optical fiber 22.

The object light transmitted through the probe optical fiber 22 is incident on the proximal end of the optical fiber 11 via the rotary joint 31. The tip of the optical fiber 11 is optically and mechanically connected to a GRIN lens 13 through a ferrule 12. The object light is incident on the GRIN lens 13 through the optical fiber 11. On a tip face of the GRIN lens 13, the deflection prism 14 is fixed, for example, by adhesion. Each of the components including the optical fiber 11, the ferrule 12, the GRIN lens 13 and the deflection prism 14 has a cylindrical shape, and is accommodated in an outer sheath 15 forming an outer appearance of the OCT probe 10. More precisely, the deflection prism 14 has a shape formed by cutting one end of a column by a plane intersecting with an axial direction to have an angle. The cut surface is coated with aluminum to form a reflection surface. The outer sheath 15 is formed of flexible materials so that the OCT probe 10 can be inserted into a lumen.

The object light bends by approximately 90° at a point where the reference axis AX intersects with the reflection surface of the deflection prism 14, while being converged by the GRIN lens 13. The bent object light transmits through the outer sheath 15 and is emitted toward a side wall of the lumen T. At least the periphery of the deflection prism 14 is filled with silicon oil to suppress loss of light amount due to the difference in refractive index.

The deflection prism 14 is fixed with respect to the optical fiber 11. When the entire configuration defined from the optical fiber 11 to the deflection prism 14 rotates about the reference axis AX in accordance with driving of the radial scan motor 32, the object light scans on the lumen T in the circumferential direction.

As the low coherence light, near infrared light having a property of propagating through a living tissue relative to visible light is used. The object light reaches a portion near the surface layer of the lumen T, and is strongly reflected or scattered at a point near a light-collecting point. Then, a part of the object light is incident on the GRIN lens 13 via the deflection prism 14. Returning light which has entered the GRIN lens 13 returns to the fiber interferometer 21 via the optical fiber 11, the rotary joint 31 and the probe optical fiber 22.

The reference light emerges from the tip of the reference optical fiber 26 through the reference optical fiber 26, and is incident on the lens 27. The lens 27 converts the reference light into collimated light, and the collimated light emerges from the lens 27. The dach mirror 28 causes the collimated light emerging from the lens 27 to be incident again on the lens 27. In order to make an optical path length of the reference light changeable, the dach mirror 28 is supported to be able to freely move in the optical axis direction (a direction of an arrow in FIG. 1) by a driving mechanism not shown. The reference light sent back to the lens 27 returns to the fiber interferometer 21 via the reference optical fiber 26.

In the fiber interferometer 21, measurement of an interferometric signal using the principle of a low coherence interferometer is performed. Specifically, in the fiber interferometer 21, an interferometric signal is obtained only when optical path lengths of the object light returned from the probe optical fiber 22 and the reference light returned from the reference optical fiber 26 are equal to each other. The intensity of the interferometric signal is determined depending on a degree of reflection or scattering of the object light occurred at a particular position of the lumen T (the optical path length of the object light) corresponding to the position of the dach mirror 28 (the optical path length of the reference light), and becomes particularly strong at the optical path length near the light-collecting point.

The fiber interferometer 21 outputs, to the signal processing circuit 24, the interferometric signal corresponding to an interference pattern of the object light and the reference light. The signal processing circuit 24 executes a predetermined process for the inputted interferometric signal, and assigns a pixel address to the interferometric signal depending on a scanning position of the interferometric signal. The scanning position in the circumferential direction of the lumen T is identified by a driving amount of the radial scan motor 32, and the scanning position in the depth direction of the lumen T is identified by the driving amount of a drive motor (not shown) of the dach mirror 28.

The signal processing circuit 24 performs buffering, into a frame memory not shown on a frame by frame basis, for a signal of an image constituted by a spatial arrangement of point images represented by the interferometric signals in accordance with the assigned pixel addresses. The buffered signal is swept out from the frame memory at predetermined timing, and is outputted to an information processing terminal 41 of a display device 40. The information processing terminal 41 executes a predetermined process for the inputted signal and converts the inputted signal into a video signal, and displays an image near the surface layer of the lumen T on a monitor 42.

Next, three examples of a concrete configuration of the OCT probe 10 are explained. In examples 1 to 3, concrete configurations for reducing a frictional resistance between the optical fiber 11 and the outer sheath 15 in order to smoothly transmit the rotation torque produced by the proximal side of the optical fiber 11 to the tip side of the optical fiber 11 are proposed. According to the examples 1 to 3, since a transmission property of the rotation torque can be improved without using an expensive torque wire which is used in a conventional configuration, the rotation period of the deflection prism 14 becomes stable and thereby the fluctuation of the scanning speed can be suppressed. Furthermore, in the example 3, in order to make the light-collecting point stable while suppressing the swinging motion of the tip portion of the optical fiber 11 in the outer sheath 15, a concrete configuration for achieving a weight balance of internal components of the outer sheath 15 is proposed.

### Example 1

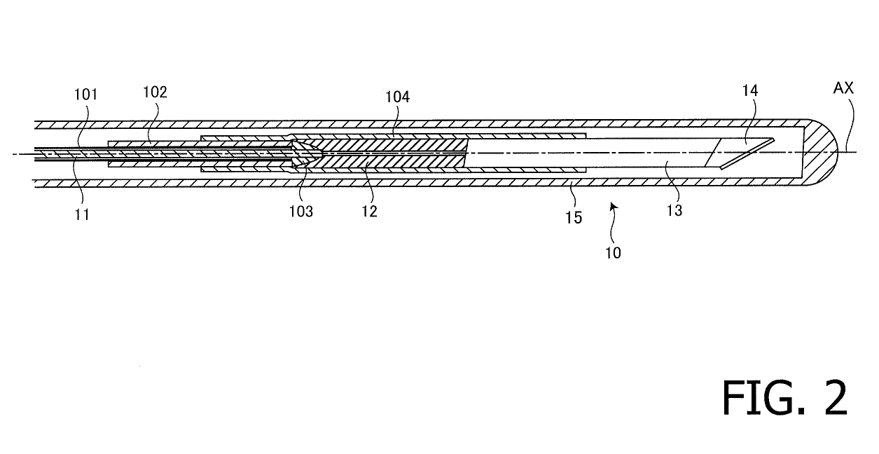

FIG. 2 illustrates an internal configuration of the OCT probe 10 according to the example 1 of the invention. To an outer circumferential surface of a PTFE (Polytetrafluoroethylene) inner sheath 101 covering a portion near the tip of the optical fiber 11 according to the example 1, an FEP (Fluorinated Ethylene Propylene) heat shrinkable tube 102 is pressurized and joined. After pressure joining of the FEP heat shrinkable tube 102, the tip surface of the optical fiber 11 is adhered to the proximal surface of the ferrule 12 with a thermosetting adhesive 103. To the outer circumferential surface extending from a portion near the tip of the heat shrinkable tube 102 to a portion near the proximal end of the GRIN lens 13 via the ferrule 12, an FEP heat shrinkable tube 102 is pressurized and joined, so that the adhered point is strengthened.

The inventors of the invention understand that a primary factor that obstructs smooth transmission of the rotation torque produced on the proximal side of the optical fiber 11 to the tip side of the optical fiber 11 is a frictional force between the optical fiber 11 and the outer sheath 15. As a concrete solution, the example 1 employs the configuration which is advantageous in regard to smooth transmission of the rotation torque by covering the whole optical fiber 11 with the PTFE inner sheath 101 having a low degree of frictional resistance. Since the frictional resistance with respect to the outer sheath 15 reduces, loss of torque is small even when the PTFE inner sheath 101 contacts the inner circumferential surface of the outer sheath 15 during rotation thereof, and therefore the PTFE inner sheath 101 is able to smoothly rotate. In addition to a low degree of friction property, the PTFE inner sheath 101 has features such as a wear resistance and a chemical resistance, and therefore is suitable as a component of the OCT probe 10.

### Example 2

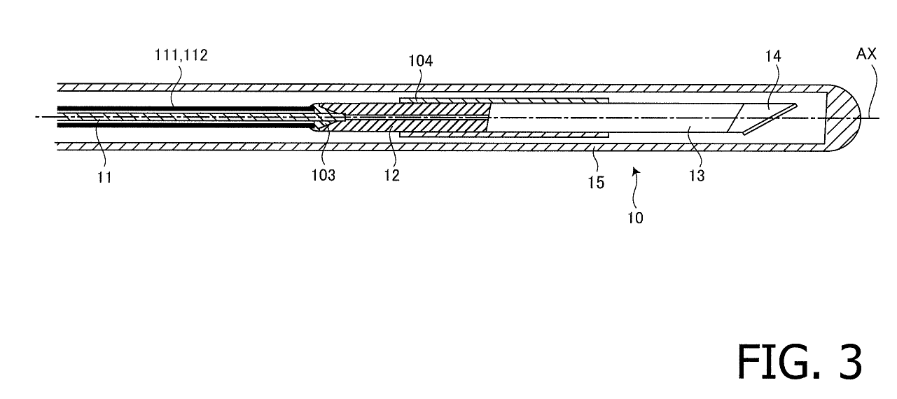

FIG. 3 illustrates an inner configuration of the OCT probe 10 according to the example 2 of the invention. In each example explained below, to elements which are the same as or similar to those of the example 1, the same reference numbers are assigned, and explanations thereof will be simplified or omitted.

Since a coating surface of the fluorocarbon resin, such as PTFE exemplified in the example 1, has a low frictional coefficient, almost no frictional resistance is caused. In the example 2, in place of PTFE, the whole outer circumferential surface expending from the tip to the proximal end of the optical fiber 11 is covered with a PI coat 111 as primary coating, and is further covered with a PFA coat 112 as secondary coating. In the example 2, since no clearance is secured between the optical fiber 11 and a coating layer, the rotation torque of the radial scan motor 32 is transmitted more smoothly and effectively to the tip side of the optical fiber 11. In the example 2, the optical fiber 11 and the ferrule 12 after covering with the PFA coat 112 are sufficiently adhered and fixed by only the thermosetting adhesive 103. For this reason, in the example 2, the FEP heat shrinkable tube 102 is omitted from the components, and the pressure joining area of the FEP heat shrinkable tube 102 is restricted to the GRIN lens 13 and the ferrule 12 only.

### Example 3

The inventors of the invention understand that a primary factor that causes the swinging motion of the tip portion of the optical fiber 11 in the outer sheath 15 is a shift between the barycenter of the component fixed to the tip of the optical fiber 11 and the rotation center axis (the reference axis AX) of the optical fiber 11. In the example 2, of the components accommodated in the outer sheath 15, components other than the GRIN lens 13 and the deflection prism 14 are arranged such that barycenters thereof coincide with the rotation center axis (reference axis AX) of the optical fiber 11. In other words, the barycenters of the GRIN lens 13 and the deflection prism 14 shift from the reference axis AX. For this reason, in the example 3, a barycenter adjustment member 121 is added to the configuration shown in the example 2.

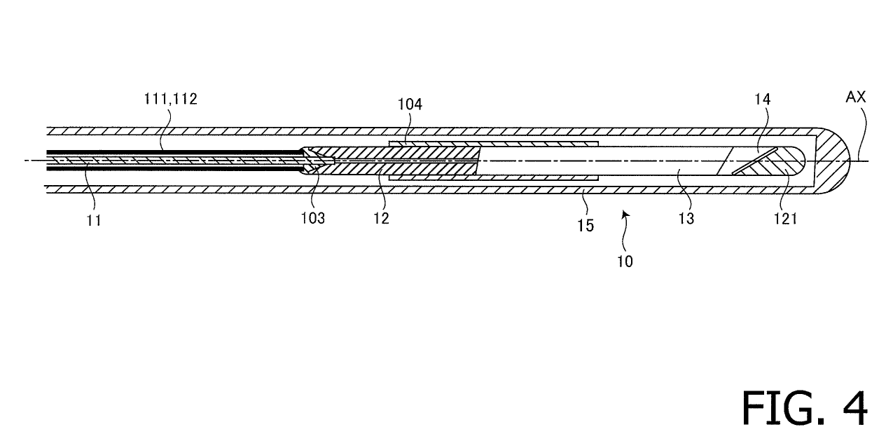

FIG. 4 is illustrates an inner configuration of the OCT probe 10 according to the example 3 of the invention. As shown in FIG. 4, the OCT probe 10 according to the example 3 has the same configuration as that of the OCT probe 10 according to the example 2 excepting that the barycenter adjustment member 121 is adhered and fixed to the back side of the reflection surface (on which the low coherence light is incident) of the deflection prism 14.

The GRIN lens 13, the deflection prism 14 and the barycenter adjustment member 121 are made of the same materials or made of materials having substantially the same specific gravity. The combined barycenter of these three components is on the reference axis AX. Since the combined barycenter of all the components (the ferrule 12, the GRIN lens 13, the deflection prism 14, the barycenter adjustment member 121 and the FEP heat shrinkable tube 102) adhered to the tip of the optical fiber 11 is on the rotation center axis, the tip portion of the optical fiber 11 stably rotates approximately on the reference axis AX. Since the position of the deflection prism 14 is also stable on the reference axis AX, the focal point is also stable. For this reason, the problem that the focal point produced when the tip portion of the optical fiber 11 causes the swinging motion fluctuates can be effectively suppressed, and thereby it becomes possible to obtain a fine tomographic image.

The volume, material and specific gravity of the barycenter adjustment member 121 are not limited as long as the combined barycenter of the GRIN lens 13 and the deflection prism 14 is located on the reference axis AX and the rotation movement thereof in the outer sheath 15 is not hampered.

There is a concern about an erosion phenomenon by cavitation when a component is rotated at a high speed in a fluid having a high degree of viscosity, such as silicon oil. For this reason, the barycenter adjustment member 121 is formed, based on a cylindrical shape having substantially the same diameter as that of the GRIN lens 13 and the deflection prism 14, by cutting a proximal end thereof by a plane forming an angle with respect to the axis direction. The angle of the cut surface formed with respect to the axis direction for the barycenter adjustment member 121 is the same as that of the deflection prism 14. The deflection prism 14 and the barycenter adjustment member 121 are adhered such that they are coaxial. Therefore, edges of the both components (the edge of the reflection surface of the deflection prism 14 and the edge of the proximal end surface of the barycenter adjustment member 121) do not appear on the outer shape contour. Furthermore, the tip of the barycenter adjustment member 121 is formed to have a semispheric shape. That is, since no edge appears on the outer shape contour, there is no part having a large fluid resistance during rotational motion, and thereby occurrence of cavitation can be effectively suppressed.

Since the barycenter adjustment member 121 is adhered to the deflection prism 14, the barycenter adjustment member 121 also has the function of protecting the reflection surface of the deflection prism 14.

The foregoing is the explanation of the embodiment of the invention. The invention is not limited to the above described configuration, and can be varied within the scope of the technical concept of the invention. For example, in addition to the OCT system of TD-OCT (Time Domain OCT) type, the invention can be applied to an OCT system of FD-OCT (Fourier Domain OCT) type, such as SD-OCT (Spectral Domain OCT) type or SS-OCT (Swept Source OCT) type.

1. An OCT probe, comprising:
a flexible tube;
an optical fiber that transmits object light and is supported in the flexible tube to be able to freely rotate about an axis of the optical fiber;
an objective optical system that is fixed to a tip of the optical fiber and includes a condensing optical system which condenses the object light emerging from the optical fiber, and a deflection optical element which irradiates a subject with the object light by deflecting the condensed object light; and
a barycenter adjustment member that is fixed to the objective optical system and causes a combined barycenter of the objective optical system and the barycenter adjustment member to be situated on the axis of the optical fiber.

2. The OCT probe according to claim 1, wherein the condensing optical system, the deflection optical element and the barycenter adjustment member are made of a same material or of materials having a same specific gravity.

3. The OCT probe according to claim 1,
wherein:
the deflection optical element is a deflection prism which is formed such that at least an end of a column is cut by a plane forming a certain angle with respect to an axis direction and a cut surface of the column is processed to be a reflection surface; and
the barycenter adjustment member is formed such that:
the barycenter adjustment member is based on a cylindrical shape having substantially a same diameter as that of the deflection prism;
a tip of the barycenter adjustment member has a semispheric shape;
the barycenter adjustment member has a proximal end surface formed to be cut by a plane forming the certain angle with respect to an axis direction of the barycenter adjustment member; and
the proximal end surface is adhered and fixed to a back side of the reflection surface so that the deflection prism and the barycenter adjustment member become coaxial with each other.

4. The OCT probe according to claim 1, wherein at least a part of an outer circumferential surface of the optical fiber is covered with a fluorocarbon resin coat.

5. The OCT probe according to claim 4,
wherein the fluorocarbon resin coat is a PTFE (Polytetrafluoroethylene) coat or a multilayer coat in which a PI (Polyimide) coat and a PFA (Polyfluoroalkoxy) coat overlap with each other.
